(19) United States
(12) Patent Application Publication
(10) Pub. No.: US 2004/0158193 A1
(43) Pub. Date: Aug. 12, 2004

(54) MEDICAL APPARATUS USING SELECTIVE GRAPHICAL INTERFACE

(75) Inventors: Tuan Bui, Green Oaks, IL (US); Doron Levitas, Chicago, IL (US)

Correspondence Address: MATTHEW J. GRYZLO, 311 S. WACKER DRIVE, 53RD FLOOR, CHICAGO, IL, 60606-6622, US

(73) Assignee: Baxter International Inc., Deerfield, IL (US)

(21) Appl. No.: 09/248,057

(22) Filed: Feb. 10, 1999

(51) Int. Cl. A61M 31/00

(52) U.S. Cl. 604/65; 700/90; 128/DIG. 13

(57) ABSTRACT

### Abstract of the Disclosure

A programmable medical device, such as an infusion pump, is programmed or configured to display, or make, as active, only those keys which can provide valid input during each particular programming or operational step. Similarly, a remote controller for controlling a programmable medical device is programmed or configured to display, or make, as active, only those keys which can provide valid input during each particular programming or operational step.

### Detailed Description of the Invention

### Background of Invention

This invention relates generally to a programmable medical device and a controller for controlling a medical device, and more particularly, to a medical device and a controller having a selective graphical interface to display relevant input information to a user.

A programmable medical device is a device which is used to administer medical treatment to a patient, monitor a patient's condition or assist in the diagnosis of a patient's condition. Examples of programmable medical devices which administer treatment to a patient include infusion pumps and respirators. Examples of programmable medical devices which monitor a patient's condition include vital sign monitors and apnea monitors. Examples of programmable medical devices which assist in the diagnosis of a patient's condition include blood analyzers.

Of these various programmable medical devices, the infusion pump is probably the most common. An infusion pump is a programmable medical treatment device which is used to administer liquid medicant to a patient. The liquid medicant is supplied from a source of medicant and pumped into the patient via an input device such as a catheter or other injection device. The infusion pump may be operable in various modes, such as a continuous mode in which the liquid medicant is continuously infused at a constant rate, or a ramp mode in which the rate of infusion gradually increases, then remains constant, then gradually decreases.

Typically, monitoring of an infusion pump is performed by viewing a visual display incorporated in the infusion pump. The manner in which the liquid is infused is controlled by the infusion pump, through commands input by the caregiver using the pump's input device (for example, a keypad) to the pump's processor.

Consequently, the monitoring and/or controlling of an infusion pump is performed at the same location at which the infusion pump is disposed, whether that be at the hospital, health-care facility or in the patient's home.

As the cost of health care increases, the trend is to allow the patient to leave the hospital earlier, but to continue the therapy at the patient's home. The infusion pump, as well as other medical devices such as respirators, allow the patient to receive therapy outside the hospital environment. To ensure that the therapy is properly delivered, the caregiver (or health-care professional) must have the capability to monitor and control the medical device's functions at the patient's location. In many instances the caregiver must visit the patient's home to monitor and control the medical device. Alternatively, the caregiver can remotely monitor and control the medical device.

Co-pending U.S. patent application no. 08/951,976, filed October 16, 1997, titled "Medical Apparatus With Remote Virtual Input Device", which is assigned to the assignee of this application, describes a medical apparatus for remote monitoring and controlling of a medical treatment device, such as an infusion pump. The medical apparatus described therein includes a medical device for administering a medical treatment and a remote controller. The remote controller includes a visual display of a virtual input device (for example, an image on a computer monitor) which corresponds substantially to the medical device's input device. The caregiver can control operation of the medical device either at the patient's location using the device's input device or from the remote location by activating the virtual input device. The remote controller also displays the contents of the medical device's display.

Co-pending U.S. Patent Application No. 08/691,872, filed August 2, 1996, titled "Apparatus for Monitoring and/or Controlling a Medical Device", which is assigned to the assignee of this application, describes an apparatus for remotely monitoring and controlling a medical device. In that device data can be transferred from the medical device on a real-time basis to the remote controller while the medical device is administering medical treatment, such as infusing medicant, to a patient.

Co-pending U.S. patent application no. 08/703,543, filed August 27, 1996, titled "Medical Treatment Apparatus and Method", which is assigned to the assignee of this application, describes a combination of a medical treatment device, such as an infusion pump, and one or more sensors connected to the patient, which detect the medical condition of the patient. The sensors are also connected to the medical treatment device, so that signals generated by the sensors, which indicate the patient's medical condition, can be stored as data in a memory contained within the medical treatment device. By connecting the sensors directly to the medical treatment device, such data can be safely stored within the device for later downloading by a remote monitor/controller. Such downloading can be by a protected record available only to authorized practitioners and not the casual user or patient. In this way, stored medical condition data can be protected against negligent or inadvertent corruption. Additionally, connecting the sensors to the medical treatment device makes the medical treatment device more compact for ambulatory use, requiring fewer add-on pieces.

The foregoing medical devices and their ancillary functions can be controlled remotely from, for example, the caregiver's office using a personal computer, modem and a remote communications line such as cable, radio, wireless communication or a telephone line. However, as more features are included in the medical device, controlling it becomes more complicated, whether at the hospital or remotely. Even skilled caregivers may find programming such medical devices difficult, and make mistakes requiring reprogramming or backing up steps. With all the added functionality and complexity of medical devices such as infusion pumps, there is a need to simplify controlling of both the medical device and the remote controller and to minimize the potential for mistakes. There is a need for an intuitive and easy-to-use medical treatment apparatus and method for remotely controlling such medical devices.

### Summary of Invention

In accordance with the principles of the present invention, a programmable medical device embodying the present invention, such as an infusion pump, and a controller for controlling a programmable medical device are programmed, constructed or configured to display, as active, only those keys which can provide valid input during each particular programming or operational step. For example, when a programmable medical device such as an infusion pump is first powered up, the pump runs through initialization and self-test. The pump's display outputs a message which states, "TESTING." During this initialization and self-test phase since no input is needed from the user and no keys should be activated, all keys are unlit or otherwise indicated as being inactive. For example, if the medical device includes a touch-sensitive screen which displays an image of a keypad, during the testing phase, the keypad area of the screen displays no keys (with the possible exception of a QUIT, OFF or EXIT key). This prevents the user from being mistakenly prompted that an input is required.

When initialization and self-test are complete, the medical device may enter either a program mode or a run mode. If in the program mode, the display ceases the "TESTING" message and displays the prompt "PROGRAM?". Since the only appropriate responses are Yes, No and Off, only the "Yes", "No" and "On/Off" keys are displayed, lit or otherwise visually indicated to be active. All other keys are unlit or otherwise indicated to be inactive.

If a particular program mode has been selected, such as a continuous pump mode, then at appropriate times, the number keys may be lit so that the user can input a numerical value for pump rate or pump time, as required. In this way, the user is prompted to select only those keys which provide a valid input; unnecessary keys are not active or shown. And, since the keys are easily identified, it reduces the time for making the appropriate choices and reduces the opportunity for incorrect inputs.

Other input devices may also be used, so long as the keys can be appropriately made to be active or inactive to insure that the user does not make an inappropriate selection. By limiting the number of choices to only those required by the programming or control step, the device is intuitive and easy to use.

A remote monitor/controller which is connected to the pump for controlling and monitoring the pump may also be similarly programmed or configured. The remote monitor/controller's display displays a virtual input device for inputting the various parameters and responding to the medical device's programming and control modes. The remote monitor/controller's display may also display an image of the medical device's display so that the user can see the values input on the pump, for instance the volume to administer.

During each mode of operation of the medical device by the remote controller, only those virtual keys which are valid or needed for operation during the particular mode are displayed, lit or otherwise distinguished from the remaining virtual keys on the virtual input device. This is similar to the operation of the programmable medical device, described above, which causes its input device to display only active keys during each phase of control or programming. The remote controller's virtual input device displays only those keys which are active during each phase of control or programming. Keys which cannot be "pressed" or activated at each programming or operational step are not displayed or otherwise are indicated to be inactive on the remote monitor/controller" s display.

A preferred embodiment is one in which the virtual input device displays only active keys. No inactive keys are displayed. In other words, the program generates images of a new set of "operative" virtual keys for each program operation, omitting all "inoperative" virtual keys. The operative or active keys can be displayed in their ordinary locations on the virtual input device, or the active keys can be redrawn in a new configuration, providing room for other information, such as messages, to be displayed. In an alternative embodiment, instead of showing only active keys on the display, all keys are shown, but the inactive keys appear as unlit or shadowed or "gray" on the display.

In one embodiment, the remote controller comprises a software routine or program which runs on a computer, and which includes a graphical interface routine or subprogram which displays the virtual input device and the active keys during program and control modes. The virtual keys may be activated by the user using a mouse or the computer keyboard. A light pen or touch screen directly on the monitor may also be used to identify and select a particular key or keys, for example. In another embodiment, the remote controller may be a stand-alone device which includes a controller, monitor and electronic circuitry for providing the graphical interface displaying the virtual input device and active keys. As with the programmable medical device, by limiting the available choices of virtual input keys to the user the remote controller is easier to use. The likelihood of potential mistakes, such as hitting incorrect key strokes and having to back up and re-enter keys for a particular step is also reduced.

### Brief Description of Drawings

Fig. 1 is a block diagram of a system including an apparatus embodying the present invention for administering medical treatment to a patient and/or monitoring the condition of the patient as well as monitoring or controlling the apparatus;

Fig. 2 is a block diagram of the electronic components of a remote monitor/controller of the apparatus shown schematically in Fig. 1;

Fig. 3 is a front view of a programmable medical device specifically of a programmable infusion pump of the apparatus shown in Fig. 1;

Fig. 4 is a block diagram of the electronic components of the programmable infusion pump shown in Fig. 3;

Fig. 5 is a flowchart of the operation of the controller of the programmable infusion pump shown in Fig. 3;

Fig. 6 is a flowchart of a mode select routine for execution on a controller of the remote monitor/controller shown in Figs. 1 and 4;

Figs. 7A and 7B illustrate portions of visual displays generated on a display of the remote monitor/controller shown in Fig. 4;

Fig. 8 is a flowchart of a display control algorithm for the controller of either the remote monitor/controller or the programmable infusion pump of Fig. 1;

Figs. 9A-9C, 10A-10C, 11A-11C, 12A-12C, 13A-13C, 14A-14C, 15A-15C and 16A-16B show sample outputs of the remote controller's display; and

Figs. 17A-17D show alternate displays which distinguish between active and inactive keys on the display of the remote controller.

### Detailed Description

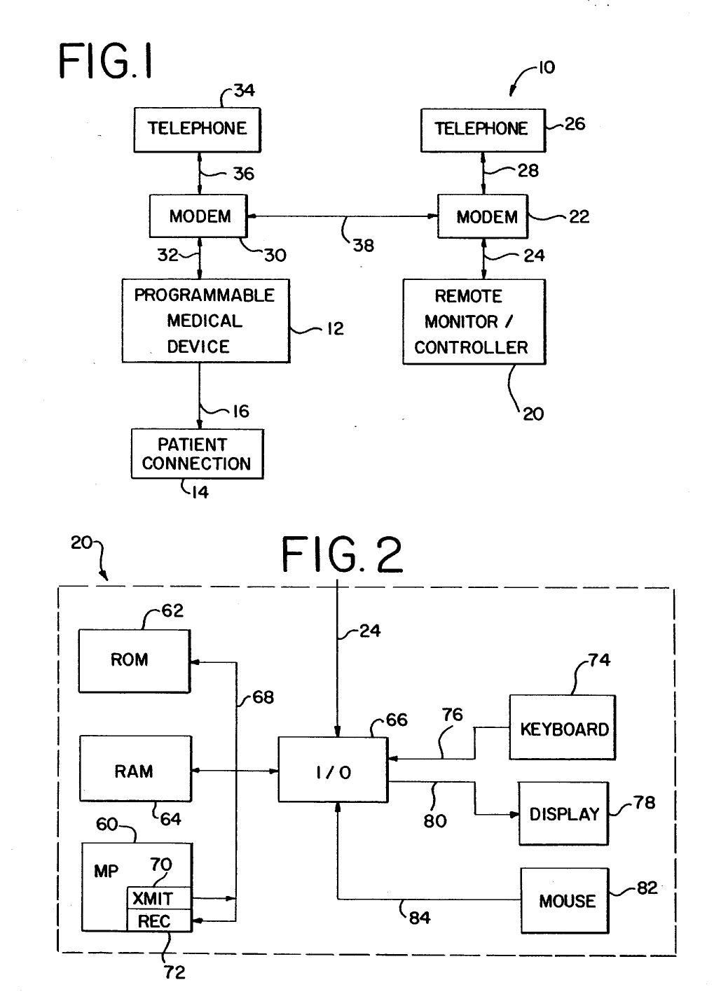

Referring to the drawings and especially to Fig. 1, an apparatus is shown therein and generally identified by reference numeral 10. Apparatus 10 includes a programmable medical device 12 and a remote monitor/controller 20. As discussed above, a programmable medical device is one which may be used to administer medical treatment to a patient, monitor a patient's condition or diagnose a patient's condition, such as infusion pumps, respirators, vital sign monitors, blood analyzers and the like. For exemplary purposes only, the invention will be described in detail with respect to a programmable infusion pump. Programmable infusion pump 12 may be used at a patient's home or in a hospital or other care facility.

Infusion pump 12 is connected to a patient connection, specifically a liquid medicant injection device in the form of a catheter 14 via a liquid conduit 16 schematically shown. The remote monitor/controller 20 is disposed for operation at a location remote from the location at which programmable infusion pump 12 is located. Remote monitor/controller 20 could be disposed in a different room of the same building in which pump 12 is disposed, or in a different building than the one in which pump 12 is disposed. Remote monitor/controller 20 is connected to a conventional modem 22 via a data link 24, and modem 22 is also connected to a telephone 26 via a voice link 28. Infusion pump 12 is connected to a conventional modem 30 via a data link 32, and modem 30 is connected to telephone 34 via a voice link 36. Modems 22, 30 are interconnected to voice and data communication via a communication link 38, which could be a telephone line, for example. The use of voice and data, in the case of remote control of the medical device, permits the caregiver to communicate with the patient while programming, monitoring or making a diagnosis. Alternatively, remote monitor/controller 20 can be linked with infusion pump 20 via wireless communications such as radio or cellular telephone. As will be described further below, either or both infusion pump 12 and remote monitor/controller 20 may include a selectable graphical interface program which enables as active only those input keys necessary for input during a particular programming or control step.

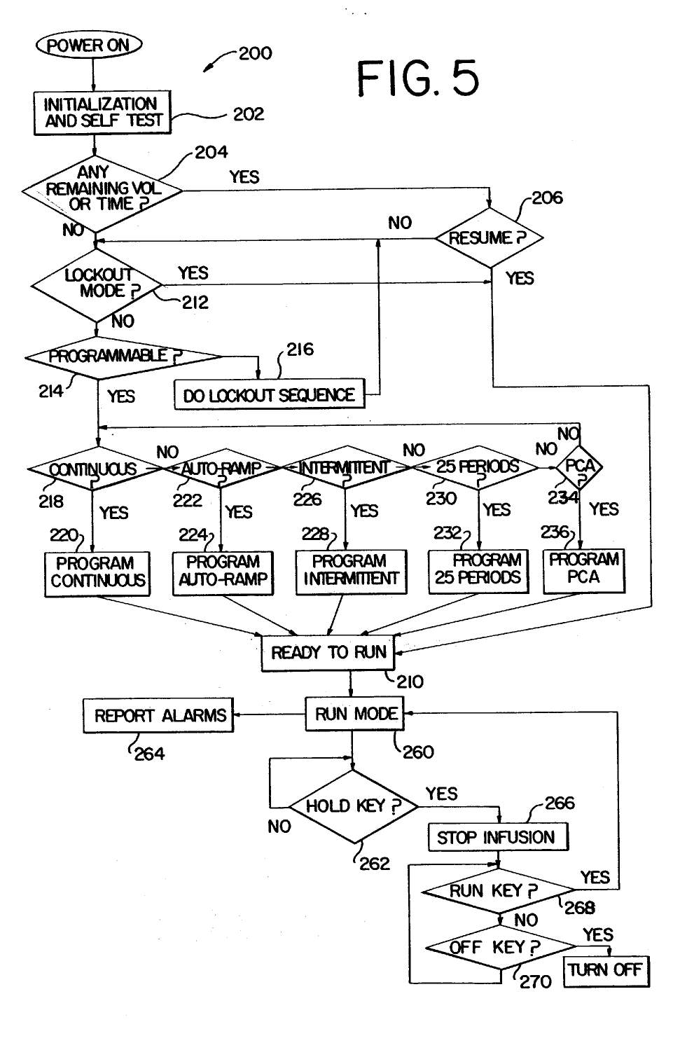

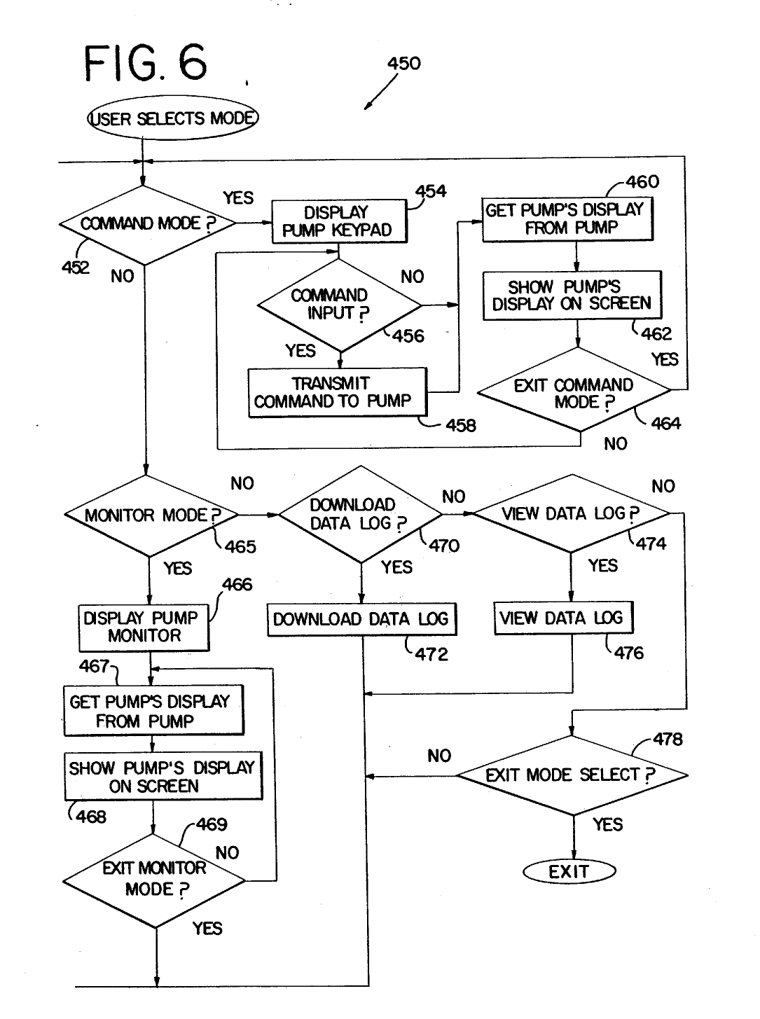

Programmable Medical Device With Selectable Graphical Interface Programmable infusion pump 12 has a housing 88 carrying an input device in the form of a keypad 90 through which a user may input data and commands and a display 92 for displaying textual messages to the user as shown in Figs. 3 and 4. Keypad 90 may also be a touch panel or screen which can display one or more or all of the keys in accordance with a selectable graphical interface program. For example, when programmable infusion pump 12 is powered off, except for the "On/Off" key, no keys are displayed or the keys are displayed in shadow with no backlighting. Alternatively, when programmable infusion pump 12 is powered on, only a blank screen may be displayed (a separate power on/off switch could be provided external to the display in this embodiment) . In another embodiment, keypad 90 may be incorporated into display 92, in which case display 92 is of a sufficient size to display both the input "keys" and data. Display 92 may be an LCD screen or other apparatus which is responsive to touch inputs, such as a touch sensitive screen or a display screen activated by radiation sensors.

Programmable infusion pump 12 includes a controller 100, an electrically programmable read-only memory (EPROM) 102 having a built-in I/O interface 102a, a nonvolatile RAM 104, a real-time clock 106 and display 92, all of which are interconnected by a communications bus 108 as shown in Fig. 4 and positioned within the housing 88. Controller 100 may be a microprocessor or other digital control device, such as an ASIC, gate array or programmable logic device. Display 92 has a backlight 110 which is selectively activated by an enable signal from controller 100. The enable signal is carried on a backlight control line 112 interconnecting controller 100 and backlight 110. Both RAM 104 and real-time clock 106 are connected to a battery 114 which supplies power to them in the absence of system power. Controller 100 has a data transmit buffer 116 and a data receive buffer 118 connected to communications bus 108.

Controller 100 controls the medicant infusion rate by periodically transmitting a control signal to an amplifier circuit 120 via a pump as amplifier signal line 122 to cause the amplifier to drive a pump motor 124 which drives a pumping mechanism 126, such as a rotary pump wheel or other type of peristaltic pump (not shown) adapted to engage with a portion of the flexible liquid conduit 16 (Figure 1) connected to the catheter 14 and apply pumping force thereto. The rotary pump wheel delivers a peristaltic pumping action to flexible liquid conduit 16 to move liquid through it.

Controller 100 receives periodic inputs from a shaft encoder (SE) sensor 130, which is disposed on the shaft of pump motor 124. Shaft encoder sensor 130 may be a two-phase motion sensing encoder which provides a two signal output to controller 100. The rotational speed of pump motor 124 and its direction of rotation are determined by controller 100 based on the rate and phase relationship between the two signal outputs.

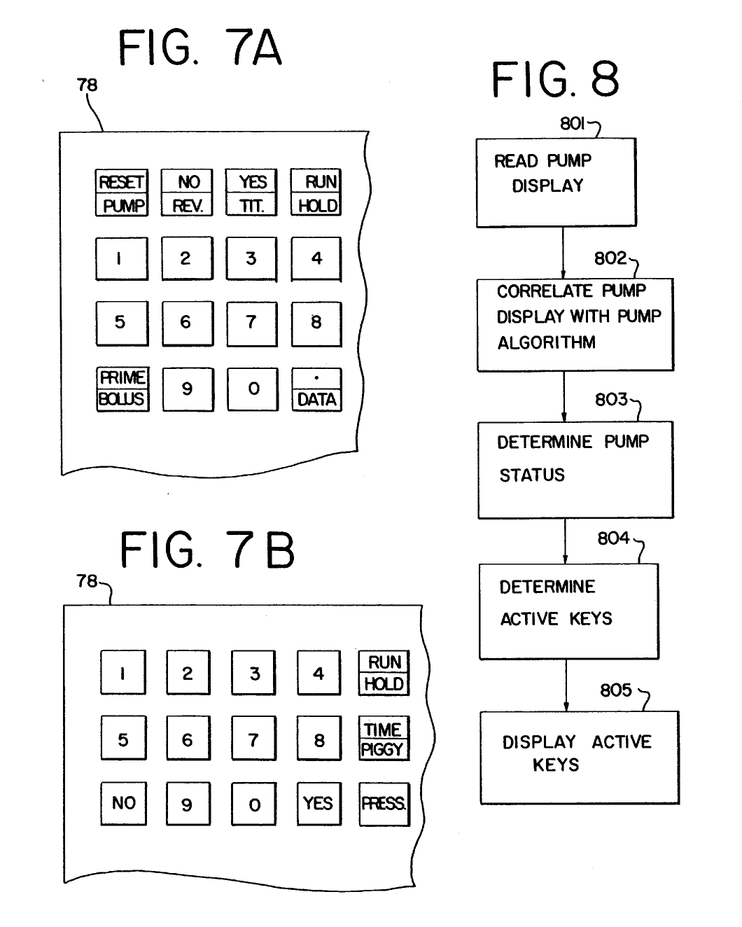

The operation of programmable infusion pump 12 is controlled by a computer program comprising routines stored in EPROM 102 and executed by controller 100. A flowchart 200 of the overall program execution in controller 100 is illustrated in Fig. 5. A separate program or subprogram controlling the selectable graphical interface, i.e. which input keys to display or activate on the pump display, is also stored in EPROM 102. The selectable graphical interface or key display program interacts with the pump's operational program to display or highlight only those keys needed for the particular pump status or operation at a particular time.

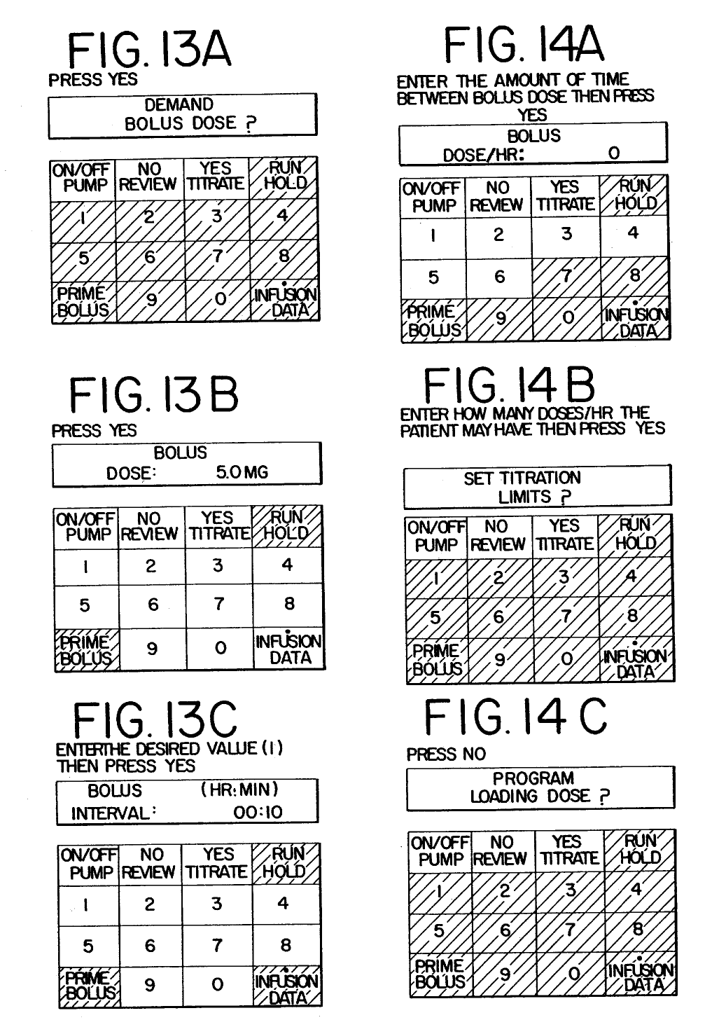

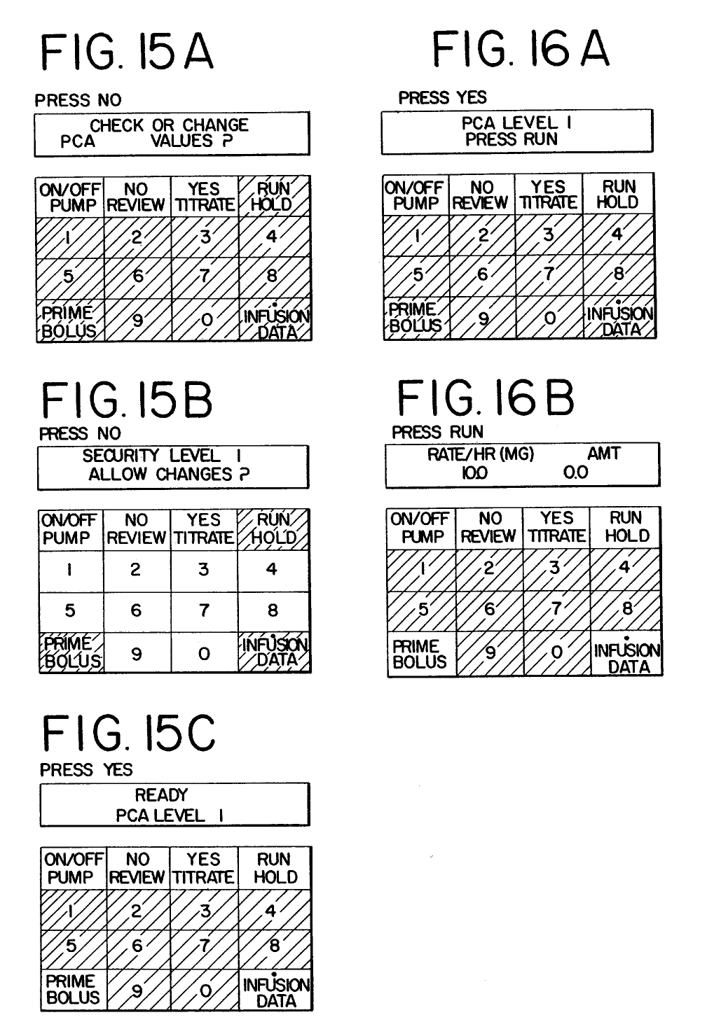

A flowchart of the interaction of the selectable graphical interface program with the pump's operational program is shown in Fig. 8. Note that this algorithm may also be used in controlling the display of the remote monitor/controller 20 as described below. Referring to Fig. 8, in step 803 the selectable graphical interface program determines the pump status. In step 804, using the pump status, the selectable graphical interface program determines the display of the appropriate keys for the particular pump status, for example program, test, data input and the like. Finally, in step 805, the selectable graphical interface program causes the appropriate active keys to be displayed, highlighted or otherwise made active. In the case of a pump with an LCD touch panel which displays the keys on the panel, the selectable graphical interface program displays only those active keys. Examples of such screens, similar to those seen on a remote monitor/controller, are shown in Figures 9A-17D. In the case of a pump with a separate input keypad, the selectable graphical interface program sends signals to the individual keys inactivating all keys except those required for operation based on the particular pump status.

Referring to Fig. 5, when programmable infusion pump 12 is turned on, at step 202 programmable infusion pump 12 is initialized and a test of the pump operation is performed. Pump 12 may be turned off temporarily during an infusion, in which case pump 12 may continue the infusion when it is turned back on, as described below. At step 204, if there is any remaining volume of liquid to be infused by the pump or any additional time remaining for an infusion, which could be the case where the pump was temporarily halted during an infusion, the program branches to step 206, where the user is asked, via a message displayed on display 92, whether the previous infusion should be resumed. If the user answers yes, the program branches to a ready-to-run step 210. If the previous infusion is not to be resumed, the program branches to step 212.

Programmable infusion pump 12 has a lockout mode in which the user may be prevented from programming the infusion parameters, such as the volume to be infused or the rate of infusion. For example, programmable infusion pump 12 could be programmed by a health care professional to deliver a particular infusion having a particular flow profile, flow rate and volume to be infused. After programming that infusion, the health care professional could place the pump 12 in lockout mode, which would prevent the patient from changing any of the infusion parameters. At step 212, if pump 12 has been previously placed in lockout mode, the program branches directly to the ready-to-run step 210, bypassing all programming steps.

At step 212, if pump 12 is not in lockout mode, the program branches to step 214, at which point the program prompts the user, via the display 92, to input whether the patient should be allowed to program programmable infusion pump 12 during the subsequent infusion. If programmable infusion pump 12 is not to be programmable, the program branches to step 216 where a lockout sequence is performed by requesting the user to input which infusion modes should be locked out. If pump 12 is to be programmable by the patient, the program bypasses step 216.

Infusion pump 12 has five basic modes of infusion: 1) a continuous mode in which the pump delivers a single volume at a single rate; 2) an auto-ramp mode in which the pump delivers liquid at a rate that gradually increases to a threshold rate, stays constant at the threshold rate, and then gradually decreases; 3) an intermittent rate in which the pump delivers discrete liquid volumes spaced over relatively long periods of time, such as a liquid volume every three hours; 4) a custom mode in which the pump can be programmed to deliver a unique infusion rate during each of twenty-five different time periods; and 5) a pain-controlled analgesic (PCA) mode during which the pump will periodically infuse boluses of analgesic in response to periodic requests by the patient.

At step 218, programmable infusion pump 12 generates on display 92, the prompt "Continuous?" to the user. In this embodiment of pump 12 with a separate keypad 90, the selectable graphical interface program causes only the Yes, No and On/Off keys to be active. If the user desires to use the pump 12 in its continuous mode, the user answer "Yes" via keypad 90, and the program branches to step 220. In step 220 the continuous mode is programmed by the user by entering a number of infusion parameters, such as the desired infusion rate, the volume to be infused, etc. During these programming steps, the selectable graphical interface program allows only the numeric keys and the On/Off keys to be active.

At step 218, if the user does not want to use the continuous mode, the user answers "No" and the program branches to step 222. Steps 222-236 are generally the same as steps 218 and 220, except that the user may be prompted for different infusion parameters, depending on which of the five possible infusion modes is selected. In each case, the selectable graphical interface program renders inactive those keys on keypad 90 not required for user input.

After completion of one of the steps 220, 224, 228, 232 or 236, the program branches to the ready-to-run step 210. When the user presses the "Run" key, programmable infusion pump 12 enters the run mode 260 and infuses the patient with a liquid medicant in accordance with the infusion mode selected at one of the mode steps 218, 222, 226, 230, 234 and the infusion parameters entered at one of parameter input steps 220, 224, 228, 232, 236. Pump 12 remains in the run mode 260 until the "Hold" key is pressed, as determined at step 262. Upon the occurrence of an alarm condition, an alarm is reported at step 264. At step 262, if the hold key is pressed, the infusion is stopped at step 266, and pump 12 waits for the run key to be pressed at step 268 or the on/off switch to be turned off at step 270.

Summarizing the operation described above, if the pump 12 is to be utilized in lockout mode, a health care professional turns the pump on, programs the desired infusion mode at one of steps 220, 224, 228, 232, 236, and then turns the pump off. The programmed infusion parameters will be retained in RAM memory 104. The health care professional would then turn the pump back on, press the "No" key in response to the "Programmable?" prompt at step 214, enter the lockout information at step 216, and then turn the pump off again. When the patient subsequently turned on programmable infusion pump 12 to perform the infusion, the program would proceed from step 212 directly to the ready-to-run step 210, which would prevent the patient from altering the infusion parameters.

If the lockout mode was not utilized, the health care professional or the patient could turn the pump on, program the desired infusion mode, and then press the "Run" key to start the infusion without ever turning the. pump off.

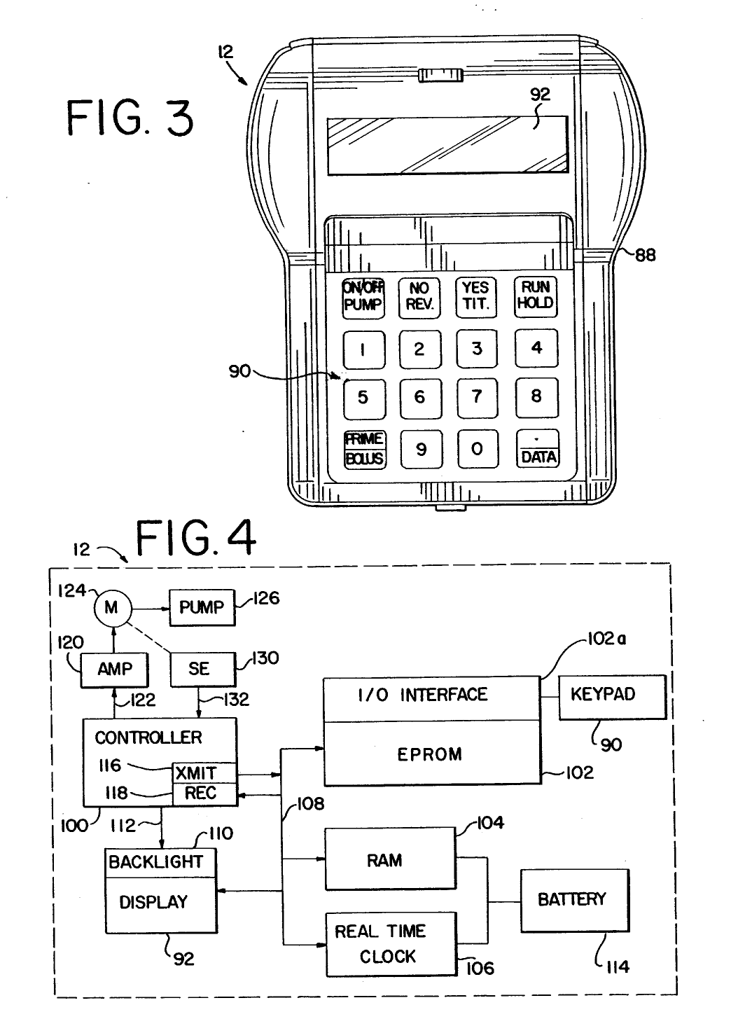

Remote Monitor/Controller With Selectable Graphical Interface Referring to Fig. 2, remote monitor/controller 20 includes a microprocessor (MP) 60, a read-only memory (ROM) 62, a random access memory (RAM) 64, and an input/output circuit 66, all of which are interconnected by an address/data (communications) bus 68. Microprocessor 60 may be a microprocessor or other digital control device, such as an ASIC, gate array or programmable logic device. Microprocessor 60 has a transmit buffer (XMIT) 70 for transmitting data bytes and a receive buffer (REC) 72 for receiving data bytes. Remote monitor/controller 20 has a keyboard 74 connected to I/O circuit 66 via a line 76, a display device 78, such as a CRT or LCD panel, connected to I/O circuit 66 via a line 80, and an input device, such as an electronic mouse 82, connected to I/O circuit 66 via a line 84. Remote monitor/controller 20 can also include one or more disk drives, such as a hard disk drive or a floppy disk drive. Remote monitor/controller 20 can be a stand-alone device as described above or a software routine or program operating on a personal computer, using many of the personal computer's hardware components to provide the above-described functions.

Remote monitor/controller 20 allows four basic functions to be performed, including: 1) controlling medical device 12; 2) monitoring the operation of medical device 12; 3) transferring data from medical device 12 to remote monitor/controller 20 and 4) viewing data. The user may perform one of these functions by selecting an operational mode displayed on display device 78 of remote monitor/controller 20 via mouse 82 or keyboard 74. These modes include: 1) a command mode (or program mode) in which a health care professional at remote monitor/controller 20 may transmit command signals to medical device 12 to control its operation; 2) a monitoring mode (also used for diagnosis or troubleshooting) in which medical device 12 will continually transmit the contents of its display 92 to remote monitor/controller 20; 3) a download data mode in which infusion data is transferred from medical device 12 to remote monitor/controller 20; and 4) a view data mode in which the infusion data may be viewed on display 78 of remote monitor/controller 20.

A selectable graphical interface program, similar to the one described with respect to medical device 12 is stored in ROM 62 and executed by microprocessor 60. Referring to Fig. 8, in step 801 the selectable graphical interface program reads the pump display. Then in step 802 it correlates the display with a pump algorithm. The pump algorithm determines which keys are required for appropriate input from the user depending on the particular pump status or programming step. Then the selectable graphical interface program determines the pump status in step 803. In step 804, using the pump status, the selectable graphical interface program retrieves the display of the appropriate keys for the particular pump and status. Examples of such screens as would be seen on the remote monitor/controller are shown in Figures 9A-17D and described further below. Finally, in step 805, the selectable graphical interface program causes the appropriate active keys to be displayed or highlighted.

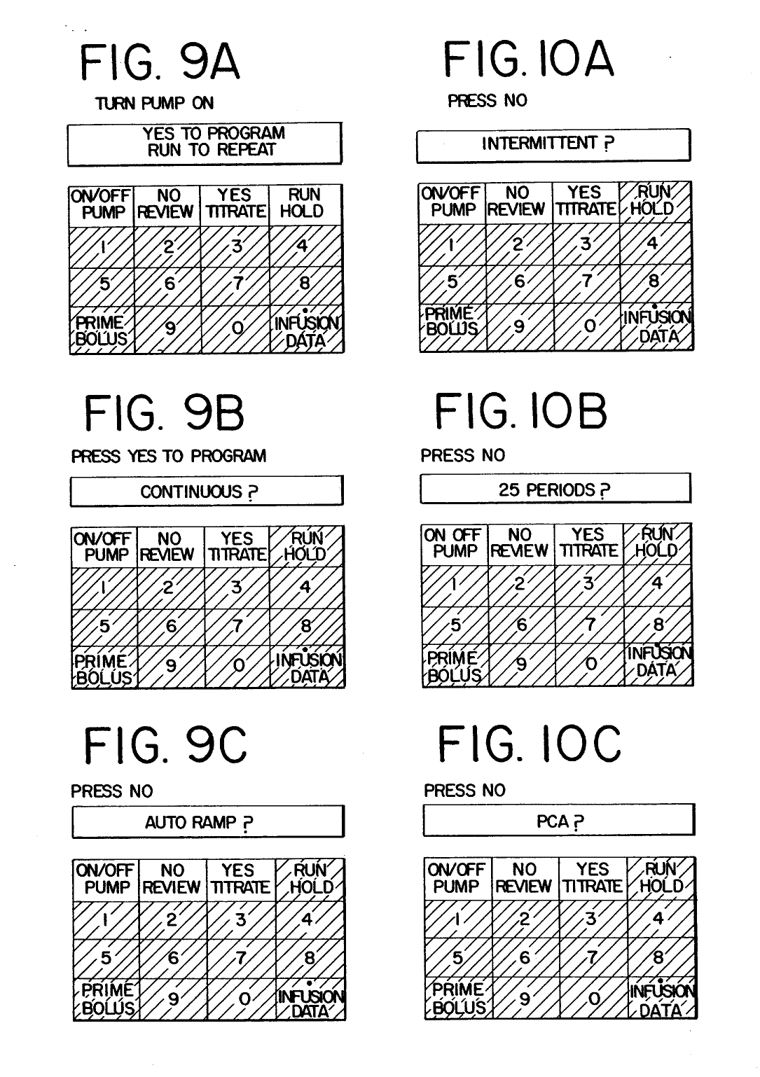

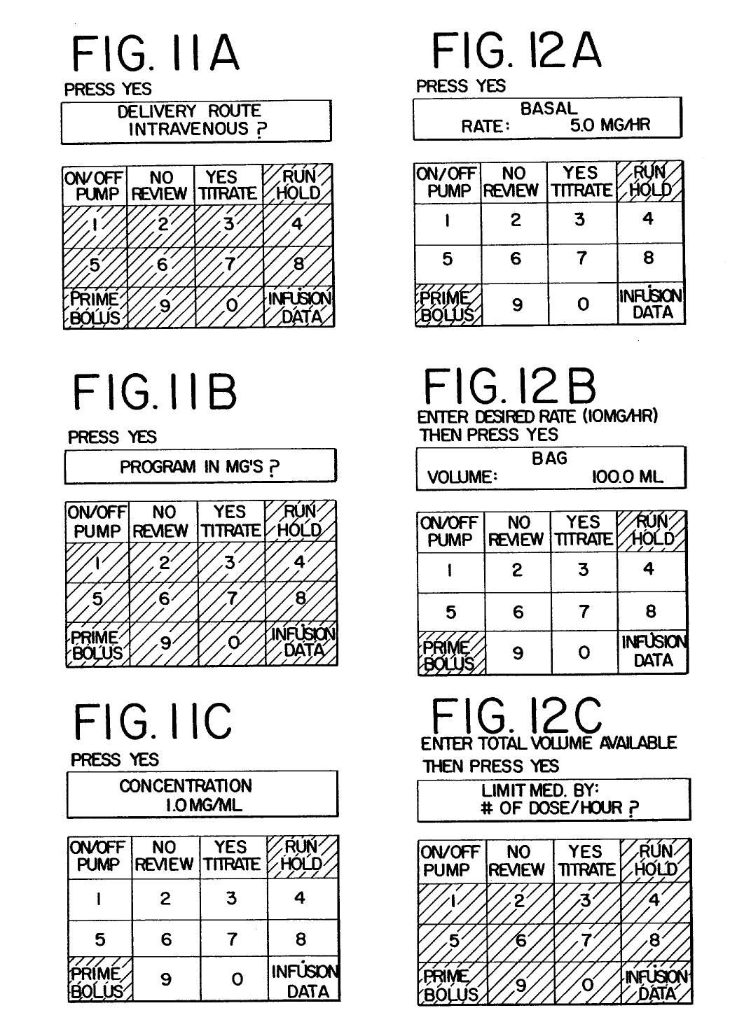

Referring to Fig. 6, at step 452, if the user selected the command mode described above, the routine branches to step 454 where the selectable graphical interface program causes a display of keypad 90 of medical device 12 to be shown on display device 78. The display shown during step 454 includes a plurality of virtual entry keys having a spatial configuration substantially the same as the entry keys of keypad 90 of the particular infusion pump type which is connected to remote monitor/controller 20. An example of such a visual display is shown in Fig. 7A. It should be noted that the display of the complete virtual entry keys is only momentary. Thereafter, only those keys which are required for user input are displayed or otherwise indicated to be active.

It should also be noted be noted that the virtual keypad shown in Fig. 7A (the complete virtual keypad) is the same as the actual keypad 90 of medical device 12 (shown in Fig. 3), except that the On/Off key of medical device 12 is replaced with the Reset key in the virtual key display. Where a different type of medical device having a different keypad is attached to remote monitor/controller 20, that particular keypad is displayed on display device 78. An example of a different keypad for a different medical device is shown in Fig. 7B. Various virtual keypad configurations for different medical devices can be stored in the memory of remote monitor/controller 20, each virtual keypad having a medical device type code associated with it. Remote monitor/controller 20 initially determined the type of medical device to which it is attached (via a routine, for example, as disclosed in co-pending application no. 08/951,976, filed October 16, 1997, titled "Medical Apparatus With Remote Virtual Input Device", it can retrieve from memory and display the corresponding virtual keypad for the medical device.

After the selectable graphical interface program displays the appropriate active virtual keys, the health care professional may control the operation of medical device 12 by selecting any of the active virtual keys with mouse 82. Other ways of selecting the active keys could also be used, such as, receiving inputs from a touch-sensitive screen or a display activated by radiation sensors. Medical device 12 responds to commands entered via its keypad 90 and/or to commands generated from remote monitor/controller 20. At steps 456 and 458, any commands entered by the health care professional are transmitted to medical device 12, and at steps 460 and 462, the display information of medical device 12 is transferred to the remote monitor/controller 20 and displayed on display device 78 of remote monitor/controller 20. At step 464, if the user exits the command mode, the routine branches back to step 452.

At step 465, if the health care professional selected the monitor mode, the routine branches to step 466 where a visual display of medical device display 92 is also shown on display device 78. At step 467, the contents of medical device display 92 are transferred to remote monitor/controller 20, and at step 468 those contents are displayed in the virtual display generated at step 466.

At step 469, if the user exits the monitor mode, the routine branches back to step 452; otherwise, the routine branches back to step 467 so that the contents of pump display 92 are continuously shown on display device 78 at step 468 (display 92 of medical device 12 changes in accordance with the medical device operation so that the medical device operation can be monitored by viewing display 92) . Step 467 may be accomplished, for example, by transmitting a medical device display request to medical device 12 (via steps similar to steps 416-420 described above). If the health care professional inputs a request to download data from medical device 12 to remote monitor/controller 20 as determined in step 470, the routine branches to step 472 where the data transfer is accomplished, for example, as described in co-pending application no. 08/951,976, filed October 16, 1997, titled "Medical Apparatus With Remote Virtual Input Device". If the user inputs a view data log request as determined at step 474, the routine branches to step 476 where data previously downloaded at step 472 can be viewed on display device 78 of remote monitor/controller 20. The user may exit the mode select routine 450 via step 478.

Examples of Selectable Graphical Interface Displays Figures 9A - 17D show example screens of a remote monitor/controller during various programming and operation steps for a PCA profile in which only active keys are displayed. Note that in these figures, the entire virtual keypad is shown, with inactive keys shown in shadow or gray tones and active keys as white background keys. Referring to Fig. 9A, the display portion of remote monitor/controller 20 (i.e. the portion which mimics what is displayed on display 92 of medical device 12) shows "YES to Program RUN to Repeat". In the input area of the display, only the "On/Off?", "No", "YES" and "RUN" keys are displayed as active. All other keys are shown in shadow as being inactive. After the user presses "Yes", the next screen (Fig. 9B) prompts the user by asking "Continuous?" In the input area of the display, only the "On/Off?", "No" and "Yes" keys are displayed as active. All other keys are shown in shadow as being inactive. After the user presses "No", the screen shown in Fig. 9C is displayed, with the same keys shown as active. Operation of the remaining screens is similar. The user presses "No" to "Auto-Ramp?" and Fig. 10A is displayed. The user presses "No" to "Intermittent?" and Fig. 10B is displayed. The user presses "No" to "25 periods?" and Fig. 10C is displayed. The user presses Yes" to "PCA?" and Fig. 11A is displayed.

There are several possible ways to deliver the drug: intravenous and subcutaneous. If the user presses "No" the screen will display "Subcutaneous?". The user presses "Yes" to "Deliver Route Intravenous?" and Fig. 11B is displayed. The user presses "Yes" to "Program in mgs?" and Fig. 11C is displayed. In Fig. 11C, all keys are available except "Run" and "Prime".

In Pain Control Analgesic (PCA) mode the user can select a basal rate which is a continuous basic rate of drug delivery and a bolus amount which is the additional drug that can be delivered on top of, or in addition to, the basal rate at specific time intervals. After setting the continuous rate, the screen prompts the user for Basal in Fig. 12A. After entering the desired rate, e.g. 10 mg/hr, and pressing "Yes", Fig. 12B is displayed. The user then enters the total volume and presses "Yes" and the screen shown in Fig. 12C is displayed. The user presses "Yes" to "Limit Med. by # of Dose/Hour?" and Fig. 13A is displayed. In response to a "Yes" to "Demand Bolus Dose?" Fig. 13B is displayed. The user enters the desired value, presses "Yes" and Fig. 13C is displayed.

After entering the amount of time between bolus doses, Fig. 14A is displayed. After setting the number of doses per hour and pressing "Yes", the screen shown in Fig. 14B is displayed. The user presses "No" to "Set Titration Limits" and Fig. 14C is displayed. The user presses "No" to "Program Loading Dose" and Fig. 15A is displayed. The user presses "No" to "Check or Change PCA Values?" and Fig. 15B is displayed. After pressing "Yes" to "Security Level 1 Allow Changes?", Fig. 15C is displayed. After pressing "Yes", Fig. 16A is displayed. Finally, the user presses "Run" and Fig. 16B is displayed and the pump begins to operate.

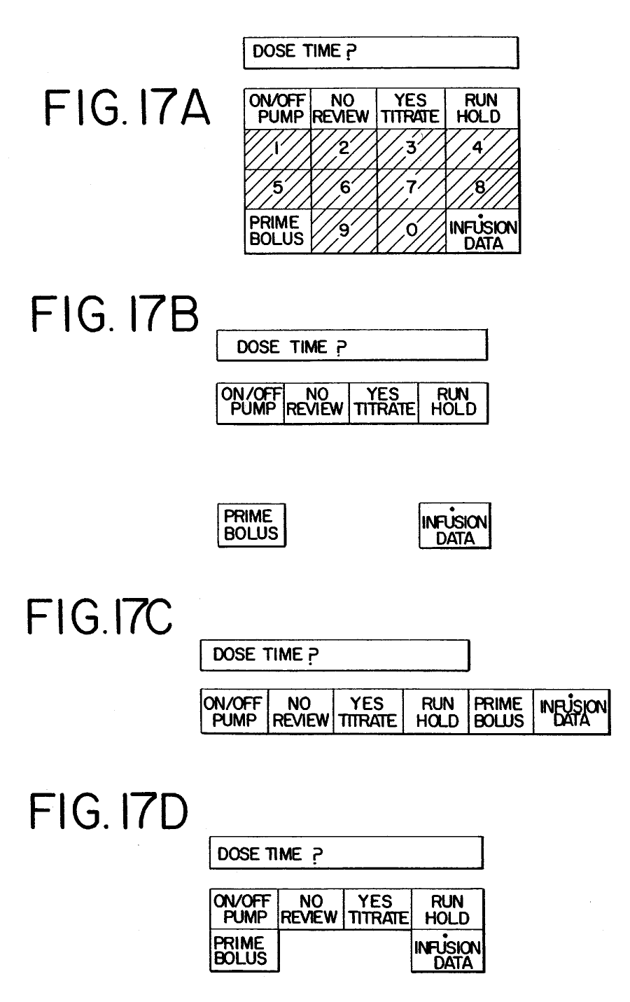

Figures 17A-17D show alternate means of presenting the active keys on the remote controller. In Fig. 17A, the non-active keys are shadowed or made less visible than the active keys. In Fig. 17B, the non-active keys are invisible, but the layout of the keys remains the same as for the pump input device. In Figures 17C and 17D the active keys are rearranged in a more prominent order.

While there has been illustrated and described a particular embodiment of the present invention, it will be appreciated that numerous changes and modifications will occur to those skilled in the art, and it is intended in the appended claims to cover all those changes and modifications which followed in the true spirit and scope of the present invention.

What is Claimed is:

1.A programmable medical device, comprising:
a display device;
an input device for allowing a user to input commands to control the programmable medical device, the input device comprising:
a routine, responsive to a status of the programmable medical device, for generating a display of a plurality of entry keys disposed in a spatial configuration and for selectively displaying on the display device only those entry keys which are required by the status for inputting commands to the programmable medical device; and
a selector for allowing a user to activate the displayed keys to allow the user to input commands to control operation of the programmable medical device.

2.The programmable medical device of claim 1 wherein said programmable medical device comprises a medical treatment device for administering a medical treatment.

3.The programmable medical device of claim 1 wherein said programmable medical device comprises an infusion pump.

4.The programmable medical device of claim 1 wherein said programmable medical device comprises a respirator.

5.The programmable medical device of claim 1 wherein said programmable medical device comprises a vital sign monitor.

6.The programmable medical device of claim 1 wherein said programmable medical device comprises an apnea monitor.

7.The programmable medical device of claim 1 wherein said programmable medical device comprises a blood analyzer.

8.The programmable medical device of claim 1 wherein the display device comprises an apparatus responsive to touch inputs or the display.

9.The programmable medical device of claim 8 wherein the display device comprises a touch sensitive screen.

10.The programmable medical device of claim 1 wherein the display device comprises an LCD.

11.A programmable medical device, comprising:
a display device;
an input device for allowing a user to input commands to control the programmable medical device, the input device comprising:
a plurality of entry keys disposed in a spatial configuration;
a routine, responsive to a status of the programmable medical device, for selectively enabling only those entry keys which are required by the status for inputting commands to the programmable medical device.

12.The programmable medical device of claim 11 wherein said programmable medical device comprises a medical treatment device for administering a medical treatment.

13.The programmable medical device of claim 11 wherein said programmable medical device comprises an infusion pump.

14.The programmable medical device of claim 11 wherein said programmable medical device comprises a-respirator.

15.The programmable medical device of claim 11 wherein said programmable medical device comprises a vital sign monitor.

16.The programmable medical device of claim 11 wherein said programmable medical device comprises an apnea monitor.

17.The programmable medical device of claim 11 wherein said programmable medical device comprises a blood analyzer.

18.The programmable medical device of claim 11 wherein the input device comprises a keypad and the routine comprises instructions for physically disabling those entry keys which are not required by the current programmable medical device status.

19.The programmable medical device of claim 11 wherein the plurality of entry keys are displayed on the display device and the routine causes selective display of the entry keys as active or inactive.

20.The programmable medical device of claim 19 wherein the inactive keys are displayed in shadow.

21.A controller for controlling a programmable medical device comprising:
a display device;
a routine, responsive to a status of the programmable medical device, for generating a display of a plurality of entry keys disposed in a spatial configuration and for selectively displaying on the display device only those entry keys which are required by the status for inputting commands to the programmable medical device; and
a selector for allowing a user to activate the displayed keys to allow the user to input commands to control operation of the programmable medical device.

22.The controller of claim 21 wherein the selector comprises a mouse.

23.The controller of claim 21 wherein the selector comprises a light pen.

24.The controller of claim 21 wherein the selector comprises a keypad.

25.The controller of claim 21 wherein the selector comprises an apparatus responsive to touch inputs.

26.The controller of claim 21 wherein the programmable medical device includes an input device having a plurality of entry keys disposed in a spatial configuration and wherein the routine causes display of the entry keys on the controller in a configuration substantially the same as the entry keys on the medical device.

27. The controller of claim 26 further comprising a memory for storing a plurality of different key configurations, each key configuration corresponding to a plurality of entry keys of a different programmable medical device.

28.The controller of claim 27 further comprising a routine for determining a type of the programmable medical device and for determining an appropriate display for each operation of the programmable medical device.

29.The controller of claim 21 wherein the controller further comprises a personal computer.

30.A medical apparatus comprising:
a programmable medical device, the programmable medical device being disposed at a first location and comprising:
an input device for allowing a user to input commands to control the medical device, the input device having a plurality of entry keys disposed in a spatial configuration; and
a remote controller for monitoring and controlling the programmable medical device, the remote controller being positionable at a second location remote from the first location but in communication therewith, the remote controller comprising:
a display device;
a routine, responsive to a status of the programmable medical device, for generating a display of a plurality of virtual entry keys disposed in a spatial configuration and for selectively displaying on the display device only those virtual entry keys which are required by the status for inputting commands to the programmable medical device; and
a selector for allowing a user to activate the displayed virtual entry keys to allow the user to input commands to control operation of the programmable medical device.

31.The apparatus of claim 30 wherein the programmable medical device comprises an infusion pump for administering a liquid medicant to a patient, the infusion pump comprising:
a liquid injection device for connection to the patient;
a conduit connected to the liquid injection device;
a pumping mechanism for pumping the liquid medicant through the conduit and into the patient via the liquid injection device; and
a controller for controlling the pumping mechanism.

32.The apparatus of claim 30, wherein the remote controller further comprises a memory for storing a plurality of different key configurations, each key configuration corresponding to a plurality of entry keys of a different programmable medical device.

33.An apparatus as defined in claim 30 wherein the remote controller further comprises: a communication device for transmitting command signals to control the operation of the programmable medical device, a monitoring device for monitoring the programmable medical device, a data transmission device for transferring data generated by the programmable medical device and a display device for viewing data generated by the programmable medical device.

34.The apparatus of claim 33 wherein each of said communication device, monitoring device, data transmission device and display device comprises a routine stored in the memory.

35.A method for controlling a programmable medical device, the programmable medical device having a display device, an input device for allowing a user to input commands to control the programmable medical device, the input device comprising: a routine, responsive to a status of the programmable medical device, for generating a display of a plurality of entry keys disposed in a spatial configuration and for selectively displaying on the display device only those entry keys which are required by the status for inputting commands to the programmable medical device; and a selector for allowing a user to activate the displayed keys to allow the user to input commands to control operation of the programmable medical device, comprising the steps of:
determining the status of the programmable medical treatment device;
selecting those entry keys which are required by the status for inputting commands to the programmable medical device; and
displaying only those entry keys which are required by the status for inputting commands to the programmable medical device.

36.A method for controlling a programmable medical device, the programmable medical device having a display device, an input device for allowing a user to input commands to control the programmable medical device, the input device comprising: a plurality of entry keys disposed in a spatial configuration; and a routine, responsive to a status of the programmable medical device, for selectively enabling only those entry keys which are required by the status for inputting commands to the programmable medical device, comprising:
determining the status of the programmable medical device;
selecting those entry keys which are required by the status for inputting commands to the programmable medical device; and
enabling only those entry key which are required by the status for inputting commands to the programmable medical device.
